(19) United States
(12) Patent Application Publication
(10) Pub. No.: US 2010/0113799 A1
(43) Pub. Date: May 6, 2010

(54) Process for the Preparation of (R) - 5 - (2-Aminoethyl) -1- (6, 8-Difluorochroman-3-YL) -1,3-Dihydroimidazole-2-Thione

(75) Inventors: Patricio Manuel Vieira Araújo Soares Da Silva, Porto (PT); Alexander Beliaev, Mindelo (PT); David Alexander Learmonth, Alfena (PT)

Correspondence Address: CONLEY ROSE, P.C., 5601 GRANITE PARKWAY, SUITE 750, PLANO, TX, 75024, US

(73) Assignee: BIAL - PORTELA & CA S.A., S. Mamede do Coronado (PT)

(21) Appl. No.: 12/525,007

(22) PCT Filed: Jan. 31, 2008
(86) PCT No.: PCT/PT2008/000005

§ 371 Date: Sep. 18, 2009

(30) Foreign Application Priority Data
Feb. 1, 2007 (GB) 0701965.6

(51) Int. Cl. C07D 233/58

(52) U.S. Cl. 548/311.4

(57) ABSTRACT

A process for making (R)-5-(2-aminoethyl)-1-(6,8-difluorochroman-3-yl)-1,3-dihydroimidazole-2-thione and pharmaceutically acceptable salts thereof, and for making intermediates useful in the formation of said compound.

### CROSS-REFERENCE TO RELATED APPLICATIONS

This application is a filing under 35 U.S.C. 371 of International Application No. PCT/PT2008/000005 filed Jan. 31, 2008, entitled “Process for the Preparation of (R)-5-(2-Aminoethyl)-1-(6,8-Difluorochroman-3-YL)-1,3-Dihydroimidazole-2-Thione,” claiming priority of Great Britain Application No. 0701965.6 filed on Feb. 1, 2007, which applications are incorporated by reference herein in their entirety.

### FIELD OF THE INVENTION

This invention relates to a process for making (R)-5-(2-aminoethyl)-1-(6,8-difluorochroman-3-yl)-1,3-dihydroimidazole-2-thione and pharmaceutically acceptable salts thereof, especially the hydrochloride salt. The invention also relates to a process for making intermediates useful in the formation of said compound, and to the intermediates, per se.

### BACKGROUND OF THE INVENTION

(R)-5-(2-aminoethyl)-1-(6,8-difluorochroman-3-yl)-1,3-dihydroimidazole-2-thione hydrochloride is described in WO2004/033447, and is useful as a medicament for treating disorders where a reduction in the hydroxylation of dopamine to noradrenaline is of therapeutic benefit. Such disorders include cardiovascular disorders, for example, hypertension, chronic heart failure, angina, arrhythmias, circulatory disorders, migraine and anxiety disorders.

### SUMMARY OF THE INVENTION

According to one aspect of the invention there is provided a process for making a compound of formula 3:

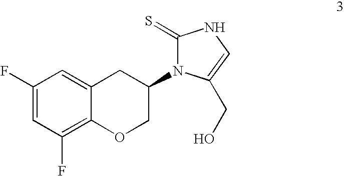

comprising reacting a compound of formula 4:

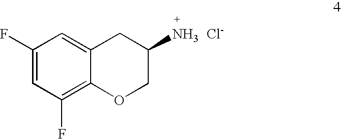

with dihydroxyacetone and a water soluble thiocyanate, and an organic acid in the presence of a solvent.

Compound 4 may be synthesised starting from L-serine methyl ester hydrochloride by condensation of its N-trityl derivative with 2,4-difluorophenol under Mitsunobu conditions followed by deprotection, ethoxycarbonylation of the resulting amino acid, Friedel-Crafts cyclization of N-protected derivative and reduction of the ethoxycarbonylamino ketone. The alkaline hydrolysis of ethyl carbamate gives 4:

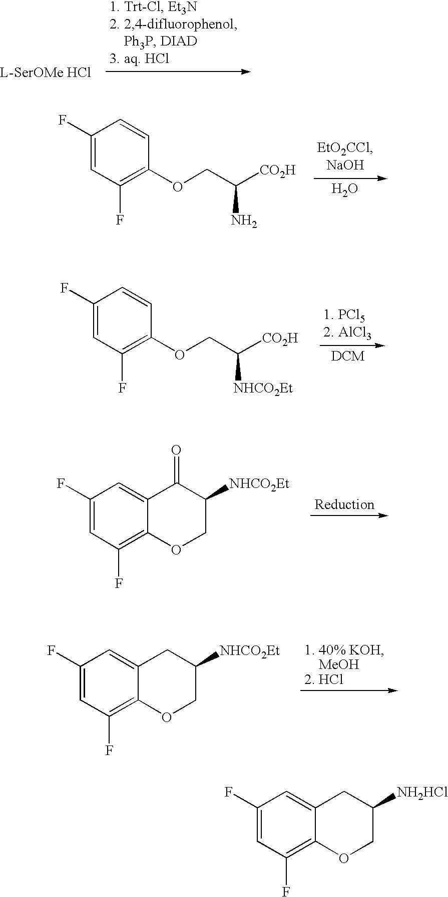

A wide variety of solvents may be used in this process, including chlorinated solvents, hydrocarbons, alcohols, esters, ethers. Preferably, the solvent is ethyl acetate.

Preferably the organic acid is a carboxylic acid, preferably a carboxylic acid having 1 to 6 carbon atoms, such as acetic acid, propionic acid and butyric acid; acetic acid is preferred. It is possible for the solvent to be the same as the organic acid.

The reaction is preferably carried out at a temperature from 40° C. to 60° C.

The water soluble thiocyanate is preferably an alkali metal thiocyanate, most preferably potassium thiocyanate.

It is preferred that the process includes the step of purifying the compound of formula 3.

According to another aspect of the invention there is provided a process for making a compound of formula 2:

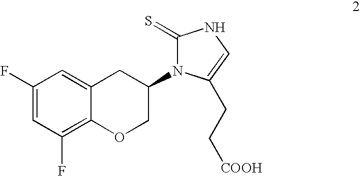

comprising reacting a compound of formula 3:

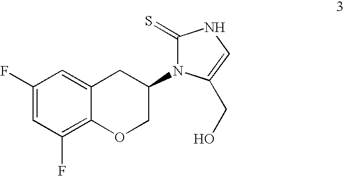

with a dialkyl malonate and a base in the presence of a solvent, wherein each alkyl group in said dialkyl malonate independently contains from 1 to 6 carbon atoms.

The base should be a stronger base than the dialkyl malonate, and is preferably an alkali metal alkoxide, more preferably an alkali metal ethoxide. Sodium ethoxide or potassium tert-butoxide are preferred.

The dialkyl malonate may suitably comprise dimethyl, diethyl or dipropyl malonate. Diethyl malonate is preferred.

The solvent may be any suitable inert solvent, but it is preferably an alcohol having 1 to 6 carbons atoms. Most preferably the solvent is ethanol.

It is preferred that the process includes the step of purifying the compound of formula 2.

### DETAILED DESCRIPTION

According to another aspect of the invention there is provided a process for making a compound of formula 1:

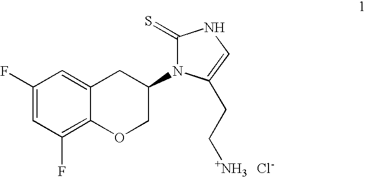

comprising reacting a compound of formula 2:

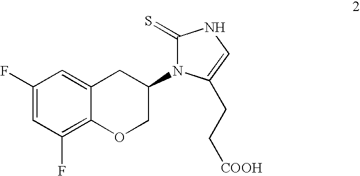

with a suitable azide in the presence of a solvent, followed by reaction with hydrochloric acid.

Preferably the azide is diphenylphosphoryl azide or an alkali metal azide, such as sodium azide.

It is to be noted that to form pharmaceutically acceptable salts other than the hydrochloride salt, an acid other than hydrochloric acid may be selected. Selection of a suitable acid and conditions is within the knowledge of the skilled person, and does not require undue experimentation. Alternatively, the salt, eg the hydrochloride salt, may be converted to the free base and isolated, or, optionally, converted to a still further pharmaceutically acceptable salt.

The solvent may be any suitable inert solvent. Preferably the solvent is a mixture of ethyl acetate and triethylamine.

According to another aspect of the invention there is provided a compound of formula 3:

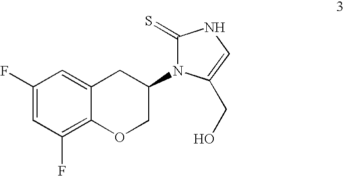

According to another aspect of the invention there is provided a compound of formula 2:

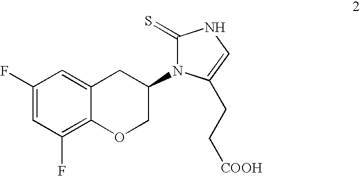

It will be appreciated that the processes according to the invention provide a method of making the compound of formula 1 starting from the compound of formula 4.

### EXAMPLES

Reference is now made to the following examples.

### Example 1

### Synthesis of (R)-1-(6,8-Difluorochroman-3-yl)-5-hydroxymethyl-1,3-dihydroimidazole-2-thione (3)

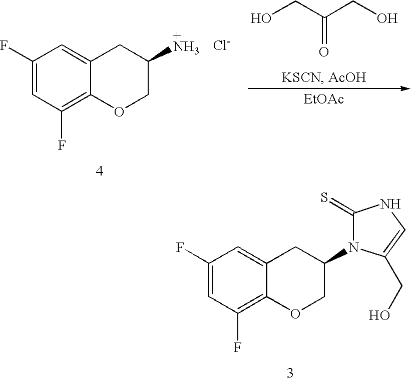

Reagents and solvents:

| | | |
|---|---|---|
| Aminochroman hydrochloride 4 | 5.10 | g (23.01 mmol) |
| Dihydroxyacetone dimer | 2.40 | g (13.4 mmol) |
| Potassium thiocyanate | 2.60 | g (26.8 mmol) |
| Acetic acid | 8 | mL |
| Ethyl acetate | 100 | mL |
| 1M H₂SO₄ | 15 | mL |
| 1N NaOH | 25 | mL |

Potassium thiocyanate was added in one portion to the suspension of 4, dihydroxyacetone dimer and acetic acid in ethyl acetate and the mixture was stirred at 50° C. for 2 hours. Heating was removed, 1M sulfuric acid was added, the mixture was stirred for 15-20 min and cooled to room temperature. Sodium hydroxide solution was added followed by sodium bicarbonate until evolution of CO₂ ceased. The organic phase was separated, washed with brine, dried over MgSO₄ and evaporated in vacuo. The residue was re-crystallised from a mixture of petroleum ether and ethyl acetate (1:1 v/v, 50 mL) in the fridge overnight, yield 5.21 g (76%), mp 166° C. (dec.).

### Example 2

### Synthesis of (R)-3-[3-(6,8-Difluorochroman-3-yl)-2-thioxo-2,3-dihydro-1H-imidazol-4-yl]propionic acid (2)

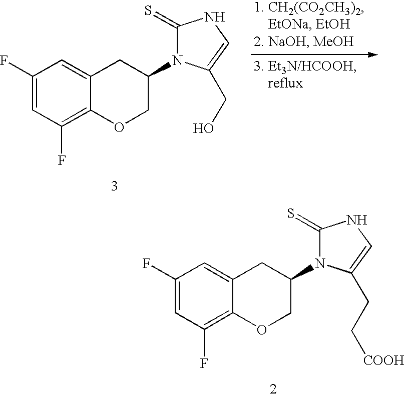

Reagents and solvents:

| | | |
|---|---|---|
| Hydroxymethyl derivative 3 | 1.59 | g (5.33 mol) |
| Sodium | 0.18 | g (7.99 mmol) |
| Ethanol abs. | 12 | mL |
| Diethyl malonate | 2.44 | mL (15.98 mmol) |
| Methanol | 27 | mL |
| Sodium hydroxide | 3.0 | g (75 mmol) |
| Water | 18 | mL |
| Formic acid | 5.3 | mL |
| Triethylamine | 7.7 | mL |

To a solution of sodium in ethanol diethyl malonate was added followed by 3 at room temperature with stiffing under nitrogen. The mixture was stirred overnight, methanol was added followed by a solution of sodium hydroxide in water. After 4 hours at room temperature organic solvents were evaporated in vacuo, the residue was diluted with water to 60 mL and the solution was washed with ethyl acetate (15 mL). The aqueous phase as acidified with 6N HCl to pH 1-2, extracted with ethyl acetate (2×30 mL). The combined extract was dried over MgSO₄ and evaporated in vacuo. The resulting oil (2.47 g) was dissolved in formic acid, triethylamine was added dropwise and the mixture was heated at 115° C. under reflux with stiffing under nitrogen for 2 hours. The solution was cooled in the ice bath and crushed ice was added to a total volume ca. 75 mL. The mixture was allowed to warm up to room temperature with stiffing, the precipitate was collected, washed with water, dried in vacuum at 40-50° C. Yield 1.38 g (76%), decomposes without melting.

### Example 3

### Synthesis of (R)-5-(2-aminoethyl)-1-(6,8-difluorochroman-3-yl)-1,3-dihydroimidazole-2-thione hydrochloride (BIA 5-453)(1)

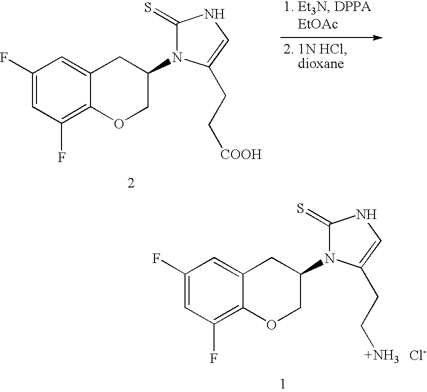

Reagents and solvents:

| | | |
|---|---|---|
| Acid 2 | 116.7 | g (342.9 mmol) |
| Triethylamine | 58.6 | mL (422.8 mmol) |
| Diphenylphosphoryl azide (DPPA) | 91.1 | mL (422.8 mmol) |
| Ethyl acetate | 1.2 | L |
| Dioxane | 2.3 | L |
| Formic acid | 115 | mL |
| 1N HCl | 460 | mL (460 mmol) |

To suspension of acid 2 in EtOAc triethylamine was added in one portion. To the clear solution formed DPPA was added at 5° C. in one portion with stirring. After 4.5 h at 5° C. the mixture was washed with cold 1N HCl (800 mL), brine (200 mL), dried (MgSO₄) and evaporated in vacuum at 27° C. The resulting suspension was diluted with dioxane (240 mL) and ether (480 mL) and left in the fridge overnight for crystallisation. The crystals were collected, washed with cold dioxane-ether mixture (1:2 v/v, 100 mL). The obtained azide (90 g after 0.5 h drying in vacuum) in the mixture of dioxane (2.3 L), 1N HCl and formic acid was heated to 60° C. with stiffing during 0.5 h, then to 75° C. in 15 min and stirred at the above temperature for 40 min. The solution was cooled to 25-30° C. and evaporated in vacuum at 45° C. to the final pressure 30 mbar. The semi-crystalline residue was re-suspended in isopropanol, evaporated to half of the initial volume, diluted with ether (1 L) and left in the fridge overnight. The crystals were collected, washed with the mixture of ether and isopropanol (2:1 v/v), dried in vacuum. The crude product (60 g) was re-crystallised by dissolving in 96% EtOH (1.1 L) under reflux, diluting with toluene (1.1 L), evaporating of the solution to half of the volume on a rotavap and crystallising overnight in the fridge. The crystals were collected, washed with toluene and dried in vacuum at 40-50° C. Yield 49.5 g (42%).

### Example 4

### Synthesis of (R)-5-(2-Aminoethyl)-1-(6,8-difluorochroman-3-yl)-1,3-dihydroimidazole-2-thione

(R)-5-(2-Aminoethyl)-1-(6,8-difluorochroman-3-yl)-1,3-dihydroimidazole-2-thione hydrochloride (9.64 g, 27.72 mmol) was dissolved in water (160 ml) at 40-45° C. with stiffing. To the resulting solution 2-propanol (64 ml) was added, the mixture was cooled to 35-38° C., dichloromethane (256 ml) was added followed by 1N NaOH (28 ml, 28 mmol) and the stirring continued for 10-15 min. Lower organic phase was separated, dried over MgSO4 and evaporated under reduced pressure to approx. 40 ml. The resulting suspension was diluted with petroleum ether (200 ml), the precipitate was collected, was with petroleum ether on the filter, dried in vacuum. Yield 7.8 g (91%), mp 192-5° C. (dec).

The free base can be converted to a desired salt using techniques known to those skilled in the art.

1. A process for making a compound of formula 3: 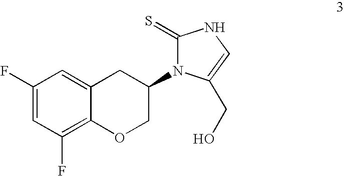
comprising reacting a compound of formula 4: 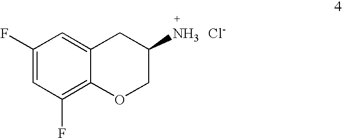
with dihydroxyacetone and a water soluble thiocyanate, and an organic acid in the presence of a solvent.

2. The process according to claim 1, wherein the solvent is ethyl acetate.

3. The process according to claim 1, wherein the organic acid is acetic acid.

4. The process according to claim 1, wherein the water soluble thiocyanate is an alkali metal thiocyanate.

5. The process according to claim 4, wherein the alkali metal thiocyanate is potassium thiocyanate.

6. The process according to claim 1, wherein the reaction is carried out at a temperature from 40° C. to 60° C.

7. The process according to claim 1, further comprising the step of purifying the compound of formula 3.

8. A process for making a compound of formula 2: 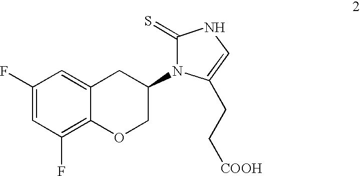
comprising reacting a compound of formula 3: 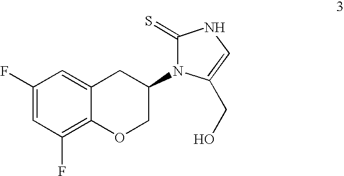
with a dialkyl malonate and a base in the presence of a solvent, wherein each alkyl group in said dialkyl malonate independently contains from 1 to 6 carbon atoms.

9. The process according to claim 8, wherein the base is sodium ethoxide.

10. The process according to claim 8, wherein the solvent is an alcohol having 1 to 6 carbons atoms.

11. The process according to claim 8, wherein the compound of formula 3 is produced by a process comprising reacting a compound of formula 4: 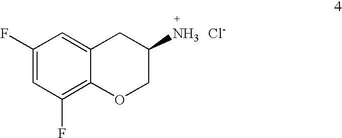
with dihydroxyacetone and a water soluble thiocyanate, and an organic acid in the presence of a solvent.

12. The process according to claim 8, further comprising purifying the compound 2.

13. A process for making a compound of formula 1: 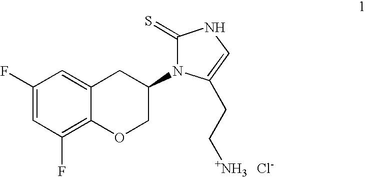
comprising reacting a compound of formula 2: 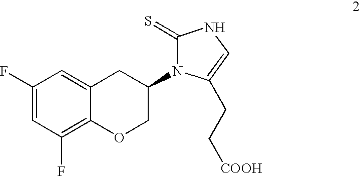
with an azide in the presence of a solvent, followed by reaction with hydrochloric acid.

14. The process according to claim 13, wherein the azide is diphenylphosphoryl azide or an alkali metal azide.

15. The process according to claim 13, wherein the solvent is a mixture of ethyl acetate and triethylamine.

16. The process according claim 13, wherein the compound of formula 2 is made by a process comprising reacting a compound of formula 3: 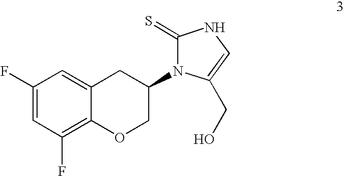
with a dialkyl malonate and a base in the presence of a solvent, wherein each alkyl group in said dialkyl malonate independently contains from 1 to 6 carbon atoms.

17. A process for forming the free base of a compound of formula 1: 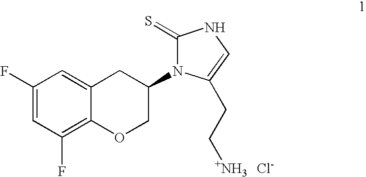
comprising reacting a compound of formula 2: 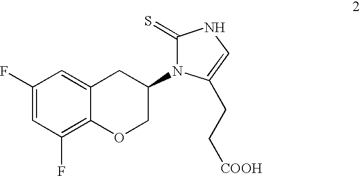
with an azide in the presence of a solvent, followed by reaction with hydrochloric acid to produce the compound of formula 1, followed by reaction with a suitable base to produce the free base of the compound of formula 1.

18. The process according to claim 17, wherein the compound of formula 2 is made by a process comprising reacting a compound of formula 3: 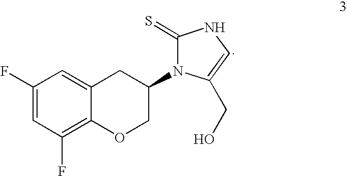
with a dialkyl malonate and a base in the presence of a solvent, wherein each alkyl group in said dialkyl malonate independently contains from 1 to 6 carbon atoms.

19. A compound of formula 3:

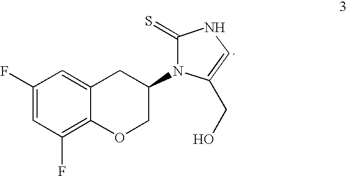

20. A compound of formula 2:

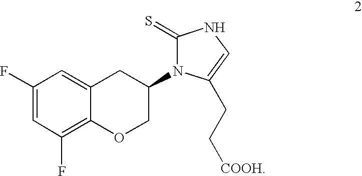

21. (canceled)

22. A compound having the formula:

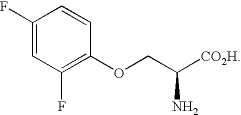

23. A compound having the formula:

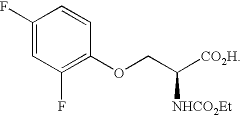

24. A compound having the formula: [chemical structure]
